(19) United States
(12) Patent Application Publication
(10) Pub. No.: US 2010/0316631 A1
(43) Pub. Date: Dec. 16, 2010

(54) Water Soluble Curcumin-Based Compounds

(75) Inventor: Ahmad Safavy, Birmingham, AL (US)

Correspondence Address: BRADLEY ARANT BOULT CUMMINGS LLP;INTELLECTUAL PROPERTY DEPARTMENT, 1819 FIFTH AVENUE NORTH, BIRMINGHAM, AL, 35203-2104, US

(73) Assignee: THE UAB RESEARCH FOUNDATION, Birmingham, AL (US)

(21) Appl. No.: 12/446,375

(22) PCT Filed: Oct. 19, 2007
(86) PCT No.: PCT/US07/22340

§ 371 Date: Apr. 20, 2009

Related U.S. Application Data
(60) Provisional application No. 60/862,057, filed on Oct. 19, 2006.

(51) Int. Cl. A61K 39/395; C07C 57/42; C07C 49/255; C07D 317/54; C07C 49/172; C07H 15/203; C07K 2/00; C07C 229/34; C07K 16/00; A61K 31/36; A61K 31/192; A61K 31/197; A61K 31/12; A61K 31/7034; A61P 35/00; A61P 29/00; C08G 73/06

(52) U.S. Cl. 424/133.1; 562/496; 568/325; 549/446; 568/328; 568/308; 568/331; 536/4.1; 530/300; 562/452; 530/387.3; 514/464; 514/568; 514/567; 514/682; 514/685; 514/679; 514/678; 514/35; 525/417

(57) ABSTRACT

The present disclosure describes the design and synthesis of a novel class of water soluble curcumin-based compounds. These water soluble curcumin-based compounds are shown to provide superior cell killing activity and exhibit increased and cell internalization solubility in aqueous solutions as compared to the free (unconjugated) curcumin. The present disclosure provides compositions for the treatment or prevention of a variety of disease states or conditions, such as but not limited to, cancer, other cell hyperproliferative disorders and chronic inflammatory conditions, said compositions comprising a water soluble curcumin-based compound.

The present disclosure claims the benefit of U.S. Provisional application No. 60/862,057, filed Oct. 19, 2006.

### FIELD OF THE DISCLOSURE

The present disclosure relates generally to the curcumin-based compounds, and specifically to water soluble curcumin-based compounds and methods of using such water soluble curcumin based compounds.

### BACKGROUND

Curcumin (diferuloyl methane or (E,E)-1,7-bis(4-hydroxy-3-methoxyphenyl)-1,6-heptadiene-3,5-dione) is a natural dietary ingredient. Curcumin is found in significant amounts in turmeric, a spice derived from the perennial herb Curcuma longa L. Curcumin can be extracted with ethanol or other organic solvents. Curcumin has been shown to exhibit a number of effects in in vitro and in vivo model systems (Aggarwal et al., Phytopharmaceuticals in Cancer Chemoprevention, 349-387; Chattopadhyay et al., Current Science', 87(1), 44-53; Campbell et al., Future Oncology, 1(3), 405-414; Ringman et al., Current Alzheimer Research, 2(2), 131-136; and Aggarwal et al, Adv Exp Med Biol. 2007; 595 :1-75). Curcumin has been reported to exert strong antioxidant and free radical-scavenging activity and inhibits lipid peroxidation, including radiation-induced lipid peroxidation. Its anti-inflammatory action may be due to its inhibitory effect on arachidonic acid metabolism via the lipoxygenase and cyclooxygnase pathways (Stoner & Muditar, (1995) J. Cell. Biochem. Suppl. 22: 169-180). Curcumin has been reported to exhibit anti-tumor and anti-apoptotic properties and to suppress the growth of a variety of cancer cell lines in the laboratory and prevent the appearance of cancers in animal studies (Araujo and Leon, 2001). Khar et al. found that curcumin induced apoptosis in leukemia, breast, colon, hepatocellular and ovarian carcinoma cell lines in vitro, but failed to demonstrate cytotoxic effects in other cancer cell lines, such as prostate (Khar et al., 2001). This difference in cytotoxic effect may be due to the poor solubility of curcumin in such model systems.

Curcumin is abundantly available in oriental diet, for example, and it is on the FDA GRAS (generally recognized as safe) list. No LD₅₀ has been reported for curcumin. Doses as high as 500-5000 mg/kg body weight have shown no toxicity when fed to animals (rats, cats, dogs, pigs and monkeys) over a period of 60 weeks. Oral, parenteral and topical administration of curcumin has been previously studied. Studies in rats where the animals were given 1 to 5 g/kg of curcumin found that 75% of the curcumin was excreted in the feces and only traces appeared in the urine. (Araujo and Leon, 2001). However despite its low toxicity, curcumin's bioavailability after oral administration is poor due to its low solubility and in vivo concentrations of curcumin that are growth inhibitory to tumor cells in vitro can be difficult to achieve through administration by the oral route. Intravenous administration of free curcumin has also been found to be ineffective to achieve significant concentrations of curcumin in tissue. Curcumin has been the subject of several clinical trials in human patients, but has only been found to have limited utility in the prevention and treatment of cancer. Such limited effectiveness may be due to the poor solubility and bioavailability of curcumin.

As a result, the widespread use of curcumin for treatment and/or prevention of human disease has been limited because of its poor water solubility, which leads to low bioavailability and problems in formulating pharmaceutical formulations. The art is currently lacking curcumin-based compounds that show improved water solubility and that are useful in formulating pharmaceutical formulations. Such soluble curcumin-based compounds would allow the development of curcumin formulations suitable for in vivo administration thereby providing increased systemic bioavailability. Thus, there remains a need in the field for soluble curcumin-based compounds (i.e., curcumin, curcumin metabolites or curcumin analogues) for use in the effective treatment of cancers human disease in vivo.

It would therefore be desirable to identify soluble derivatives of curcumin, curcumin metabolites and curcumin analogues to improve the effectiveness of such compounds in treatment and prevention strategies. Furthermore, such soluble derivatives of curcumin, curcumin metabolites and curcumin analogues would provide for effective of in vivo administration of such compounds, allowing the in vivo concentration of the compounds to be increased to a therapeutically effective level. In one embodiment, the soluble curcumin, curcumin metabolites and curcumin analogues are water soluble and are used in the treatment and/or prevention of disease states, such as but not, limited to cancer and chronic inflammation.

Therefore, it would be desirable to provide water soluble curcumin, curcumin metabolites and curcumin analogues. It would be further desirable to provide pharmaceutical formulations comprising such water soluble curcumin, curcumin metabolites and curcumin analogues exhibiting favorable properties for formulation. The use of such water soluble and pharmaceutical formulations comprising the same would increase systemic bioavailability of curcumin, thereby reducing the amount of compound required for effective treatment and prevention and resulting in a higher therapeutic index. Furthermore, since such water soluble curcumin, curcumin metabolites and curcumin analogues could be delivered in higher concentrations, the effectiveness of such compounds in prevention and/or treatment methods would be increased. The present disclosure provides novel water soluble curcumin, curcumin metabolites and curcumin analogues and provides pharmaceutical compositions comprising the same. Furthermore, the present disclosure provides methods for synthesizing such compounds. Still further, the present disclosure provides methods of treatment using the novel water soluble curcumin, curcumin metabolites and curcumin analogues and pharmaceutical formulations comprising the same.

### BRIEF DESCRIPTION OF THE DRAWINGS

So that the features, advantages and objects of the disclosure will become clear, are attained and can be understood in detail, reference is made to the appended drawings, which are described briefly below. It is to be noted, however, that the appended drawings illustrate certain embodiments of the disclosure and therefore are not to be considered limiting in their scope.

FIG. 1 shows the structure of curcumin.

FIG. 1B shows the structure of a curcumin analogue.

FIG. 2A shows the synthesis of one embodiments of a high molecular weight water soluble curcumin-based compound, in this case a curcumin-PEG conjugate, of the present disclosure.

FIG. 2B shows the synthesis of one embodiments of a low molecular weight water soluble curcumin-based compound, in this case a curcumin-PEG conjugate, of the present disclosure.

FIG. 2C shows the synthesis an alternate embodiment of a water soluble curcumin-based compound, in this case a curcumin-carbohydrate conjugate, of the present disclosure.

FIG. 2D shows the resonance-symmetric structure of curcumin.

FIG. 2E shows ¹H-NMR spectra of the ring methoxy groups of curcumin, illustrating a singlet configuration.

FIG. 2F shows ¹H-NMR spectra of the ring methoxy groups of a water soluble curcumin-PEG conjugate, illustrating a split in the chemical shifts after conjugation.

FIG. 3A shows structural confirmation of the intermediate conjugate 4 produced in FIG. 2A by MALDI-MS.

FIG. 3B shows structural confirmation of the final conjugate 5 produced in FIG. 2A by MALDI-MS.

FIG. 4A shows rate of curcumin release from one embodiment of a water soluble curcumin-based compound of the present disclosure (conjugate 5) at pH 7.4 and 37° C.; curcumin release was monitored by RP-HPLC at 280 nm.

FIG. 4B shows rate of curcumin release from one embodiment of a water soluble curcumin-based compound of the present disclosure (conjugate 8) at pH 7.4 and 37° C.; curcumin release was monitored by RP-HPLC at 280 nm.

FIG. 5 shows the effects of one embodiment of a water soluble curcumin-based compound of the present disclosure (conjugate 8) on the growth of bxPC-3 pancreatic carcinoma cells at concentrations of 1, 2.5, 5 and 10 μM as compared to unconjugated curcumin (designated CCMN). Control cells were treated with equal amounts of culture medium or DMSO, respectively.

FIG. 6 shows internalization of curcumin (left hand side) and a water soluble curcumin-based compound (conjugate 8) (right hand side) of the present disclosure in PC-3 human prostate carcinoma cells at time points of 2, 8 and 24 hours as determined by fluorescent microscopy (FITC columns). The bottom panel shows a DMSO control. Locations of the cells' nuclei are confirmed by DAPI staining and location of the internalized compound is determined by FITC staining.

### DETAILED DESCRIPTION

The present disclosure illustrates the design and synthesis of a novel class of water soluble curcumin-based compounds. These water soluble curcumin-based compounds are shown to provide superior cell killing activity as compared to the free (unconjugated) curcumin.

In one embodiment, the present disclosure provides a water soluble curcumin-based compound that demonstrates increased solubility in aqueous solutions as compared to free (unconjugated) curcumin, curcumin metabolites and curcumin analogues. Such water soluble curcumin-based compounds show increased inhibition of cell proliferation when incubated with several cancer cell lines in vitro. The synthesis of several embodiments of water-soluble curcumin-based compounds is provided in the present disclosure.

In an additional embodiment, the present disclosure provides compositions, said compositions comprising a water soluble curcumin-based compound.

In another embodiment, the present disclosure provides compositions for the treatment or prevention of a variety of disease states or conditions, such as but not limited to, cancer, other cell hyperproliferative disorders and chronic inflammatory conditions, said compositions comprising a water soluble curcumin-based compound.

In a further embodiment, the present disclosure provides methods for the treatment or prevention of a variety of disease states or conditions in a subject, such as but not limited to, cancer, other cell hyperproliferative disorders, chronic inflammatory conditions, and any disease state or condition characterized, at least in part, by up-regulated oxidation processes and/or increased generation of free radicals, said methods comprising the steps of (a) identifying a subject in need of treatment and/or prevention (b) providing a water soluble curcumin-based compound or a pharmaceutical composition comprising a water soluble curcumin-based compound as an active ingredient and (c) delivering such water soluble curcumin-based compound or a pharmaceutical composition comprising a water soluble curcumin-based compound as an active ingredient to the subject.

Other and further aspects, features, and advantages of the present disclosure will be apparent from the following description of several embodiments of the invention.

### DEFINITIONS

The term “curcumin-based compound” is meant to include curcumin, a metabolite of curcumin or an analogue of curcumin.

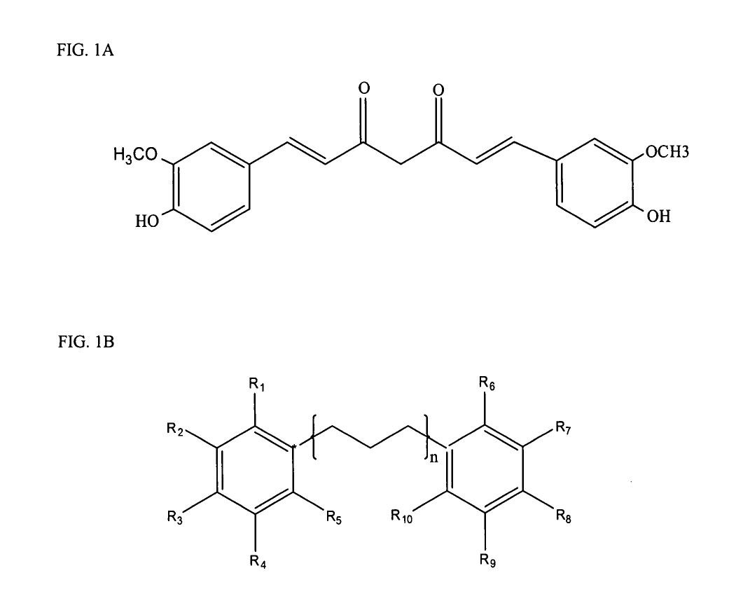

The term “curcumin” refers to a compound having the structure shown in FIG. 1A, as well as tautomers and

The term “water soluble curcumin-based compound” is meant to include any curcumin-based compound conjugated, directly or indirectly, to a solubilizing element.

The term “solubilizing element” is meant to include any compound, chemical moiety or segment of such compound or chemical moiety, associated directly or indirectly, with a curcumin-based compound that increases the solubility of the curcumin-based compound in a given solution, such as, but not limited to, an aqueous solution under physiological conditions.

The term “physiological conditions” refers to an aqueous solution having a pH from 6-8 and a temperature from 30-42 degrees Celsius.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The terms “treat” and “treating” are meant to include administering a water soluble curcumin-based compound described, either alone or as part of a pharmaceutical composition, after the onset of clinical symptoms. Such treating need not be absolute to be useful.

The terms “prevent” and “preventing” are meant to include administering a water soluble curcumin-based compound described, either alone or as part of a pharmaceutical composition, prior to the onset of clinical symptoms. Such treating need not be absolute to be useful.

The term “in need of treatment” is meant to include a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a water soluble curcumin-based compound described, either alone or as part of a pharmaceutical composition.

The term “in need of prevention” is meant to include a judgment made by a caregiver that a patient requires or will benefit from prevention. This judgment is made based on a variety of factors that are in the realm of a caregiver's expertise, but that includes the knowledge that the patient may or will become ill, as the result of a condition that is treatable by a water soluble curcumin-based compound described, either alone or as part of a pharmaceutical composition.

The term “individual”, “subject” or “patient” is meant to include any animal, including mammals, preferably mice, rats, other rodents, rabbits, dogs, cats, swine, cattle, sheep, horses, or primates, and most preferably humans. The term may specify male or female or both, or exclude male or female.

The term “prodrug,” is meant to include a compound that is rapidly transformed in vivo to a curcumin, a metabolite of curcumin or an analogue of curcumin, for example, by hydrolysis in blood. A thorough discussion of prodrugs and their synthesis is provided in T. Higuchi and V. Stella, “Prodrugs as Novel Delivery Systems,” Vol. 14 of the A.C.S. Symposium Series, Edward B. Roche, ed., “Bioreversible Carriers in Drug Design,” American Pharmaceutical Association and Pergamon Press, 1987, and Judkins et al., Synthetic Communications 26(23):4351-4367, 1996, each of which is incorporated herein by reference.

The term “therapeutically effective amount” in reference to the treating of a disease state or condition is meant to include an amount of a water soluble curcumin-based compound described, either alone or as part of a pharmaceutical composition, that is capable of having any detectable, positive effect on any symptom, aspect, or characteristics of the disease state or condition. Such effect need not be absolute to be beneficial.

### Introduction

As shown herein, the effect of a given treatment may be enhanced by the use of a water soluble curcumin-based compound. Such water soluble curcumin-based compound provides increased bioavailability of curcumin, resulting in increased therapeutic indexes and thereby lowering the dose of curcumin required to achieve a beneficial effect. Such water soluble curcumin based compounds were not previously recognized in the art.

The present disclosure provides embodiments of the water soluble curcumin-based compound by conjugating curcumin to a solubilizing element. Suitable solubilizing elements include, but are not limited to, poly(ethylene glycol) (PEG), derivatives of PEG, poly(substituted-2-oxazoline) (POZ), derivatives of POZ, an amino acid, a carbohydrate, a salt in conjunction with a component of the water soluble curcumin-based compound (such as a pharmaceutically acceptable salt), a peptide, polypeptide, a poly(amino acid), a protein, an antibody, a charged molecule, or a water-soluble natural or synthetic polymer, or any other molecule of high water solubility.

In one embodiment, PEG is used as the solubilizing element. In one embodiment, the average molecular weight of such PEG molecules may range from 100 to 5000 Da. The chemical and biological properties of PEG molecules have been extensively studied and the pharmaceutically useful characteristics of this polymer have been noted. These include aqueous as well as organic solubilities, lack of immunogenicity, and favorable blood clearance patterns and in vivo behavior. Furthermore, PEG molecules are available in a wide range of chemistries. Any form of straight-chain or branched PEG or a combination may be used as desired, including, but not limited to, mono-dispersed discrete PEG (dPEG). It shall be appreciated by those having ordinary skill in the art that various polymers can be used in addition to PEG for attachment to a curcumin-based compound described herein, such as polyoxyethylene 2-methyl-2-propenyl methyl diether, N-(2-hydroxypropyl)methacrylamide co-polymer, or polyoxyethylene allylmethyldiether In one embodiment, a straight chain PEG can be represented by the formula: X—O(CH₂CH₂O)ₙCH₂CH₂OH, (1) where n is 20 to 2300 and X is H or a terminal modification, including but not limited to a C₁₋₄ alkyl. Examples of branched PEGS are shown in US Patent Publication 20060073113.

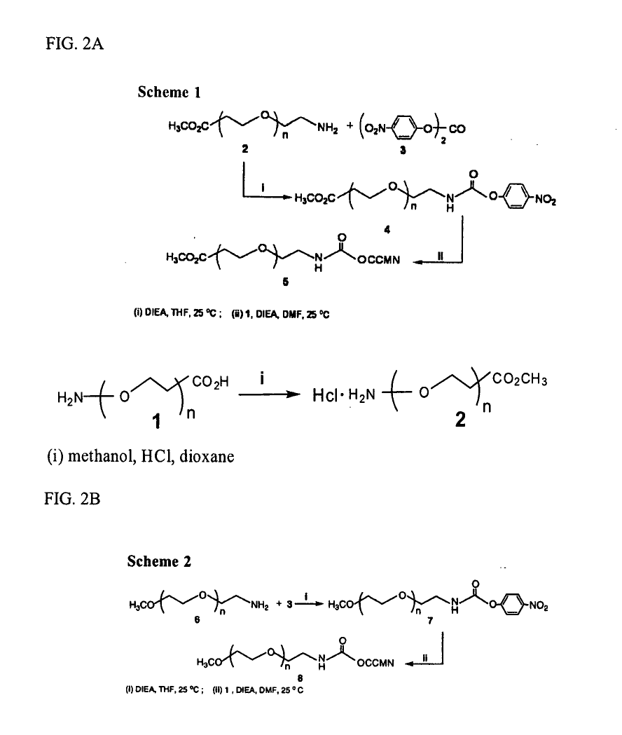

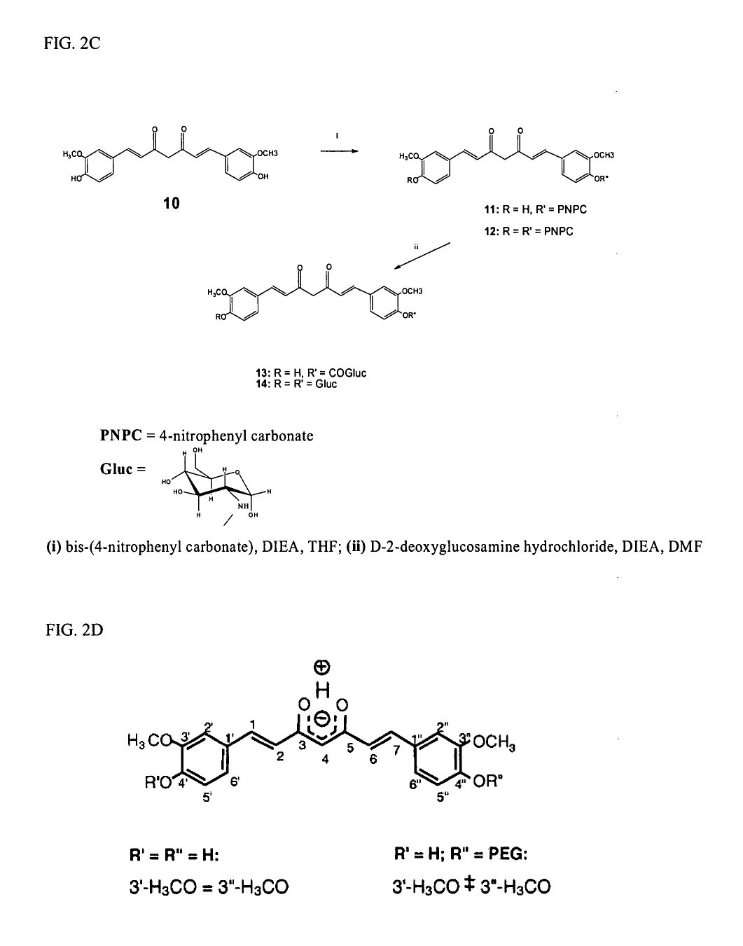

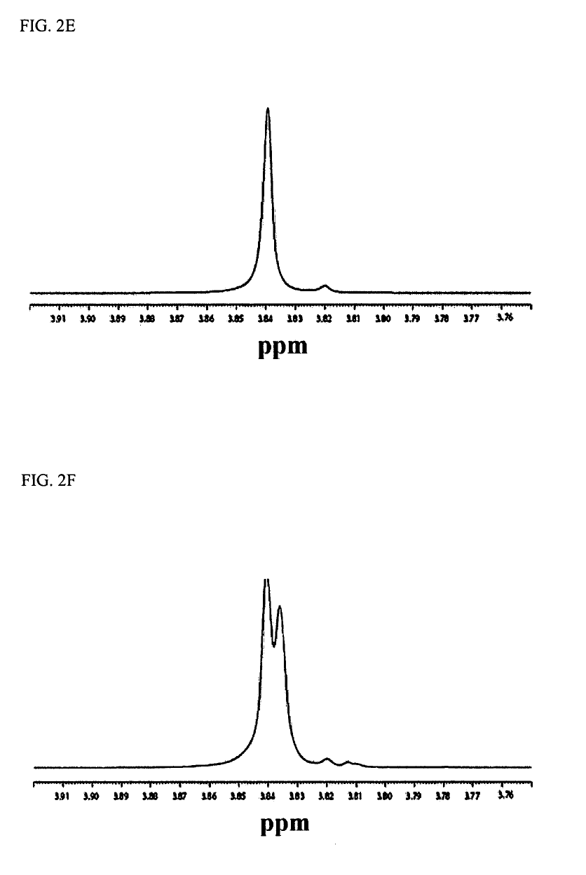

The solubilizing molecules may be positioned at any place in the conjugate desired provided that the chemistry of the functional groups present on the conjugate and/or the solubilizing agent allow for such placement. A solubilizing element may be placed at more than one location on a given conjugate. Exemplary placements of the solubilizing agent are provided in Example 2 and FIGS. 2A and 2B. However, as discussed above, the present disclosure should not be limited only to the use of PEG as a solubilizing element. The minimum requirement for a solubilizing element would be the existence of functionalities suitable for chemical coupling between the solubilizing element and curcumin, a curcumin metabolite or a curcumin analogue. A linking molecule may be used to join the solubilizing element to the curcumin, curcumin metabolite or curcumin analogue, with the linking molecule having functionalities suitable for chemical coupling between the solubilizing element and the curcumin, a curcumin metabolite or a curcumin analogue. However, a linking molecule is not required and is optional. Functional groups that may be involved in such chemical coupling (either as a part of the solubilizing element or linker) include, but are not limited to, organic amines, carboxylic acids, halides, alcohols, sulfides, hydrazides, aldehydes, and ketones. Once present, conjugation may be possible with coupling reagents as is known in the art. Exemplary types of chemical linkages which may be expected to result, include, but are not limited amide, amine, ester, ether, thioether, sulfide, disulfide, hemiacetal, acetal, ketal, hydrazide, urethane or hydrazone linkage. The functional groups and chemical bonds discussed above may be useful in coupling reactions described herein.

In certain embodiments, more than one solubilizing element may be conjugated to the curcumin-based compound. In such embodiments, more than one solubilizing element may be directly conjugated to the curcumin-based compound; alternatively, more than one solubilizing element may be conjugated to the curcumin-based compound through the use of a linker. Further, branched solubilizing elements may be used, such as but not limited to, branched PEGS; alternatively, branched linking molecules may be used to allow the conjugation of more than one solubilizing element to a curcumin-based compound. A branched PEG linker used in this invention can be a linear or branched aliphatic group that is hydrolytically stable and contains an activated moiety, e.g., an aldehyde group, which reacts with a functional group on the PEG molecule. Examples of activated, branched PEG linkers are described in U.S. Pat. Nos. 5,643,575, 5,919,455, and 5,932,462.

Curcumin has the chemical structure depicted in FIG. 1A. Curcumin may be derived from a natural source, the perennial herb Curcuma longa L., which is a member of the Zingiberaceae family. The spice turmeric is extracted from the rhizomes of Curcuma longa L. and has long been associated with traditional-medicine treatments used in Hindu and Chinese medicine. Curcumin is soluble in ethanol, alkalis, ketones, acetic acid and chloroform, however it is insoluble in water and other aqueous solutions. Curcumin is therefore lipophilic, and generally readily associates with lipids. Curcumin may also include isomers of curcumin, such as the (Z,E) and (Z,Z) isomers of curcumin, pharmaceutically acceptable salts of curcumin, prodrugs of curcumin and polymorphs and tautomers of curcumin. In certain embodiments, curcumin can be formulated as metal chelates, especially copper chelates. Other curcumins appropriate for use in the present invention will be apparent to one of skill in the art.

As used herein, the term “curcumin metabolites” includes those compounds which are metabolized by a subject from curcumin and which exhibit anti-proliferative, anti-cancer, anti-inflammatory, anti-oxidant or pro-apoptotic effects in model systems similar to that of curcumin. Known curcumin metabolites include dihydroferulic acid, ferulic acid and glucoronides of tetrahydrocurcumin and hexahydrocurcumin. Curcumin metabolites may also include isomers, such as the (Z,E) and (Z,Z) isomers, tautomers, pharmaceutically acceptable salts, prodrugs and polymorphs of curcumin metabolites. In certain embodiments, curcumin metabolites can be formulated as metal chelates, especially copper chelates. Other appropriate curcumin metabolites appropriate for use in the present invention will be apparent to one of skill in the art.

As used herein, the term “curcumin analogues” includes those compounds which due to their structural similarity to curcumin, exhibit anti-proliferative, anti-cancer, anti-inflammatory, anti-oxidant or pro-apoptotic effects in model systems similar to that of curcumin. In one embodiment, curcumin analogues which may have anti-proliferative and/or anti-cancer effects similar to curcumin include Ar-tumerone, methylcurcumin, demethoxy curcumin, bisdemethoxycurcumin, sodium curcuminate, dibenzoylmethane, acetylcurcumin, feruloyl methanecurcumin, hexahydrocurcumin, tetrahydrocurcumin, 1,7-bis(4-hydroxy-3-methoxyphenyl)-1,6-heptadiene-3,5-dione (curcuminl), 1,7-bis(piperonyl)-1,6-heptadiene-3,5-dione(piperonyl curcumin)1,7-bis(2-hydroxy naphthyl)-1,6-heptadiene-2,5-dione(2-hydroxyl naphthyl curcumin), 1,1-bis(phenyl)-1,3,8,10-undecatetraene-5,7-dione (cinnamyl curcumin) and the like (Araujo and Leon, 2001; Lin et al., 2001; John et al., 2002; see also Ishida et al., 2002). Additional curcumin analogues may include those compounds disclosed in Nicholds et al., ARKIVOC 2006 (xiii) 64-72 (ISSN 1424-6376), Ohori et al., Mol Cancer Ther 2006, 5(10) p 2563-25′71, and U.S. Pat. No. 7,060,733)

In an alternate embodiment, a curcumin analogue may have the structure of formula I shown below.

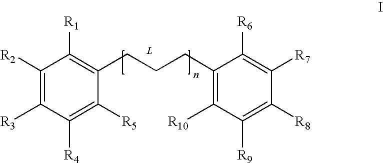

Where L is an alkyl linking moiety where n=3-20 carbons. In one embodiment, n=3-8 In one embodiment, L has the structure of formula II (diarylheptanoids) or III (diarylpentanoids).

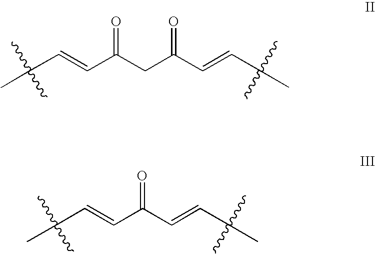

An “alkyl” is intended to mean a straight or branched chain monovalent radical of saturated and/or unsaturated carbon atoms and hydrogen atoms, such as methyl, ethyl, propyl, isopropyl, butyl, isobutyl, t-butyl, ethenyl, pentenyl, butenyl, propenyl, ethynyl, butynyl, propynyl, pentynyl, hexynyl, and the like, which may be unsubstituted (i.e., contain only carbon and hydrogen) or substituted by one or more suitable substituents as defined below (e.g., one or more oxygen atoms or halogens such as F, Cl, Br, or I; for example, one or more carbon atoms in the linker moiety L may contain single or double bonded oxygen atom).

R₁ to R₁₀ are each independently selected from the group consisting of: hydrogen, alkyl, alkoxyl, acyl, hydroxyl, amino, alkylamino, dialkylamino, carboxyl, carbamoyl, thioacyl, sulfonyl, alkoxycarbonyl. alkylaminocarbonyl, dialkylaminocarbonyl, mercapto, alkylthio and salts and esters thereof. In one embodiment, R₁ to R₁₀ are each independently selected from the group consisting of: H, OH, NO₂, and OCH₃.

An “acyl” is intended to mean a —C(O)—Rₐ radical, where Rₐ is a suitable substituent as defined below.

A “thioacyl” is intended to mean a —C(S)—Rₐ radical, where Rₐ is a suitable substituent as defined below.

A “sulfonyl” is intended to mean a —SO₂Rₐ radical, where Rₐ is a suitable substituent as defined below.

A “hydroxyl” is intended to mean the radical —OH.

An “amino” is intended to mean the radical —NH₂.

An “alkylamino” is intended to mean the radical —NHRₐ, where Rₐ is an alkyl group.

A “dialkylamine” is intended to mean the radical —NRₐR_{b}, where Rₐ and R_{b} are each independently an alkyl group.

An “alkoxyl” is intended to mean the radical —ORₐ, where Rₐ is an alkyl group. Exemplary alkoxyl groups include methoxyl, ethoxyl, propoxyl, and the like.

An “alkoxycarbonyl” is intended to mean the radical —C(O)ORₐ, where Rₐ is an alkyl group.

An “alkylsulfonyl” is intended to mean the radical —SO₂Rₐ where Rₐ is an alkyl group.

An “alkylaminocarbonyl” is intended to mean the radical —C(O)NHRₐ, where Rₐ is an alkyl group.

A “dialkylaminocarbonyl” is intended to mean the radical —C(O)NRₐR_{b}, where Rₐ and R_{b} are each independently an alkyl group.

A “mercapto” is intended to mean the radical —SH.

An “alkylthio” is intended to mean the radical —SRₐ where Rₐ is an alkyl group.

A “carboxyl” is intended to mean the radical —C(O)OH.

A “carbamoyl” is intended to mean the radical —C(O)NH₂.

Curcumin analogues may also include isomers, such as the (Z,E) and (Z,Z) isomers, tautomers, pharmaceutically acceptable salts, prodrugs and polymorphs. In certain embodiments, curcumin analogues can be formulated as metal chelates, especially copper chelates. Other appropriate curcumin analogues appropriate for use in the present invention will be apparent to one of skill in the art.

### Methods of Treatment and Prevention

The present disclosure describes the use of the water soluble curcumin-based compounds and pharmaceutical compositions containing such water soluble curcumin-based compounds in methods to treat and prevent disease states, conditions and disorders such as but not limited to, cancer, other cell hyperproliferative disorders, chronic inflammatory conditions, and any disease state or condition characterized, at least in part, by up-regulated oxidation processes and/or increased generation of free radicals. Other disease states or conditions that are characterized, at least in part, by an activity that is inhibited by curcumin may also be subject to such methods of treatment and prevention.

Cancer is the exemplary human disease state discussed below and in the Examples, but this disclosure should not be interpreted to be limited only to the treatment and/or prevention of cancer. Curcumin, curcumin metabolites and curcumin analogues have been reported to have a variety activities as discussed above and known in the art. Therefore, the curcumin-based compounds disclosed could be used in the treatment and/or prevention of disease states and conditions that are characterized, at least in part, by such activities as would be known to one of ordinary skill in the art.

Exemplary cancers that can be treated and or prevented using the methods of the present disclosure include both solid tumors and non-solid tumors such as leukemia and lymphoma. In certain embodiments, the cancer treated is prostate cancer or pancreatic cancer. The compounds of the present disclosure can be used to treat either malignant or benign cancers. Carcinomas, sarcomas, myelomas, lymphomas, and leukemias can all be treated using the compounds of the present disclosure, including those cancers which have a mixed type. Specific types of cancer that can also be treated using the compounds of the present disclosure include, but are not limited to: all forms of adenocarcinoma of the breast or prostate; all forms of bronchogenic carcinoma of the lung; myeloid; melanoma; hepatoma; neuroblastoma; papilloma; apudoma; choristoma; branchioma; malignant carcinoid syndrome; carcinoid heart disease; carcinoma (e.g., Walker, basal cell, basosquamous, Brown-Pearce, ductal, Ehrlich tumor, in situ, Krebs 2, merkel cell, mucinous, non-small cell lung, oat cell, papillary, scirrhous, bronchiolar, bronchogenic, squamous cell, and transitional cell), histiocytic disorders; leukemia (e.g., B-cell, mixed-cell, null-cell, T-cell, T-cell chronic, HTLV-II-associated, lyphocytic acute, lymphocytic chronic, mast-cell, and myeloid); histiocytosis malignant; Hodgkin's disease; immunoproliferative small; non-Hodgkin's lymphoma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; Ewing's sarcoma; synovioma; adenofibroma; adenolymphoma; carcinosarcoma; chordoma; craniopharyngioma; dysgermiinoma; hamartoma; mesenchyoma; mesonephroma; myosarcoma; ameloblastoma; cementoma; odontoma; teratoma; thymoma; trophoblastic tumor; adenocarcinoma; adenoma; cholangioma; cholesteatoma; cylindroma; cystadenocarcinoma; cystadenoma; granulosa cell tumor; gynandroblastoma; hepatoma; hidradenoma; islet cell tumor; leydig cell tumor; papilloma; sertoli cell tumor; theca cell tumor; leiomyoma; leiomyosarcoma; myoblastoma; myoma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin; angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma (e.g., Ewing's, experimental, Kaposi's, and mast-cell); neoplasms (e.g., bone, breast, digestive system, colorectal, liver, pancreatic, pituitary, testicular, orbital, head and neck, central nervous system, acoustic, pelvic, respiratory tract, and urogenital); neurofibromatosis, and cervical dysplasia), and the like. The methods of the present disclosure are useful for the treatment or prevention of cancer in all mammalian subjects, including particularly human patients.

Exemplary chronic inflammatory conditions or disease states/conditions characterized, at least in part, by up-regulated oxidation processes and/or increased generation of free radicals that can be treated and or prevented using the methods of the present disclosure include, but are not limited to, acute disseminated encephalomyelitis, alopecia greata, ankylosing spondylitis Addison's disease, antiphospholipid antibody syndrome, aplastic anemia, arthritis, autoimmune hemolytic anemia, autoimmune hepatitis, Behcet's disease, bullous pemphigoid, cardiomyopathy, celiac sprue-dermatitis, chronic fatigue immune dysfunction syndrome, chronic obstructive pulmonary disease, cicatricial pemphigoid, CREST syndrome, cold agglutinin disease, coeliac disease, Crohn's disease, diabetes mellitus (type 1), encephalitis, fibromyalgia-fibromyositis, Goodpasture's syndrome, Graves' disease, Guillain-Barre syndrome, Hashimoto's disease, idiopathic thrombocytopenic purpura, idiopathis pulmonary fibrosis, IgA nephropathy, inflammatory, inflammatory bowel disease, demyelinating polyneuropathy, juvenile arthritis, lupus erythematosus, Meniere's disease, multiple sclerosis, myasthenia gravis, opsoclonus myoclonus syndrome, optic neuritis, Ord's thyroiditis, pemphigus, pernicious anemia, primary biliary cirrhosis, Raynaud's phenomenon, rheumatic fever, rheumatoid arthritis, Reiter's syndrome, Sjögren's syndrome, sarcoidosis, scleroderma, stiff-man syndrome, Takayasu's arteritis, temporal arteritis, warm autoimmune hemolytic anemia, ulcerative colitis, uveitis, vasculitis and vitiligo.

The method of treatment comprises the steps of identifying a subject in need of such treatment, providing a water soluble curcumin-based compound of the present disclosure or a pharmaceutical composition comprising such water soluble curcumin-based compound and initiating in said subject a treatment regimen comprising administering to said subject a water soluble curcumin-based compound of the present disclosure or a pharmaceutical composition comprising such water soluble curcumin-based compound. The water soluble curcumin-based compound, whether alone or in a pharmaceutical composition, may be provided in a pharmaceutically acceptable carrier and in a therapeutically effective amount. Such administration would thereby treat the disease state or disorder. As discussed above, the treatment need not be absolute to provide benefit in the treatment methods disclosed.

The method of prevention comprises the steps of identifying a subject in need of such prevention, providing a water soluble curcumin-based compound of the present disclosure or a pharmaceutical composition comprising such water soluble curcumin-based compound and initiating in said subject a prevention regimen comprising administering to said subject a water soluble curcumin-based compound of the present disclosure or a pharmaceutical composition comprising such water soluble curcumin-based compound. The water soluble curcumin-based compound, whether alone or in a pharmaceutical composition, may be provided in a pharmaceutically acceptable carrier and in a therapeutically effective amount. Such administration would thereby prevent the disease state or disorder. As discussed above, the prevention need not be absolute to provide benefit in the treatment methods disclosed.

### Synthesis

The present disclosure also provides exemplary methods of synthesis for the water-soluble curcumin-based compounds. Examples of water-soluble curcumin-based conjugates were synthesized using the hydrophilic and biocompatible polymer PEG with high (3,500 Da) and low (700 Da) molecular weight.

An exemplary synthetic scheme for the preparation of one embodiment of a water soluble curcumin-based compound of the present disclosure is shown in FIG. 2A. A high molecular weight (average molecular weight 3,500 Da) methyl amino-PEG carboxylate 2 was converted to the activated urethane 4 through condensation with bis(4-nitrophenyl)carbonate (BNPC,3). Compound 4 was subsequently conjugated to curcumin through a direct coupling reaction under basic conditions, to afford conjugate 5 (see methods section for additional information). In an alternative approach, a high molecular weight (average molecular weight 3,500 Da) carboxylic acid-truncated amino PEG 1 (obtained from commercial sources) was esterified by methanol to the corresponding methyl amino-PEG carboxylate 2. Compound 2 is then reacted as discussed above. Compound 5 was a solid, which displayed an intense yellow color, had a solubility in water of over 1.5 g/mL, produced a viscous solution at high concentrations was completely water-soluble, and was stable when at refrigerated at 4° C. as well as at room temperature. This conjugate was a 1:1 adduct with an average CCMN content of 9.4%.

An additional exemplary synthetic scheme for the preparation of one embodiment of a water soluble curcumin-based compound of the present disclosure is shown in FIG. 2B. In order to obtain a higher drug-to-polymer (D/P) ratio the synthesis of water soluble curcumin-based compounds conjugated with smaller molecular weight PEG molecules was desired. Practically, a high D/P ratio would have the advantage of reducing the amount of the conjugate required in a given formulations. Considering that curcumin has an IC₅₀ in the micromolar range, a high concentration of the high molecular weight conjugate (such as that described above in FIG. 2A) may be required to produce a therapeutically effective dose. To this end, a water soluble curcumin-PEG was prepared by the procedure described below. In this synthesis, a 750-Da methoxy-truncated amino-PEG 6, (average molecular weight 750 Da) was coupled to a BNPC linker (3) to produce the intermediate conjugate 7. Compound 7 was conjugated to curcumin through the formation of a urethane linkage to form the final conjugate 8 (see methods section for additional information). The conjugate was also a 1:1 adduct.

The molecular weights of conjugates 5 and 8 were determined by MALDI-MS, which, in all cases, showed a curcumin-PEG ratio of unity (i.e., 1). Furthermore, the combination of MS and NMR showed one of the curcumin phenolic oxygens to be the site of conjugation to the polymeric linkers. The conjugation of one phenolic oxygen was evident from formation of a split in the chemical shifts of the neighboring methoxy protons of the curcumin moiety. These chemically equivalent protons (FIG. 2D) appeared as a singlet with a chemical shift of 3.84 ppm in the unconjugated CCMN (FIG. 2E). After conjugation, this signal was split into a pair with one peak showing a slight upfield shift of 0.01 ppm due to the attachment of the neighboring oxygen to the linker (FIG. 2F). At the same time, conjugation of the enolic oxygen of curcumin was ruled out based on both the spectroscopy data and the existence of the resonating structures shown in FIG. 2A.

In the embodiments shown in FIGS. 2A and 2B, one molecule of PEG (or other solubilizing agent) is conjugated to the curcumin molecule; however, more than one molecule of PEG (or other solubilizing agent) may be conjugated to curcumin as curcumin has more than 1 reactive site to receive a PEG molecule or other solubilizing agent. The addition of one or more additional molecules of PEG (or other solubilizing agent) may be accomplished using the methods of the present disclosure or other methods known in the art.

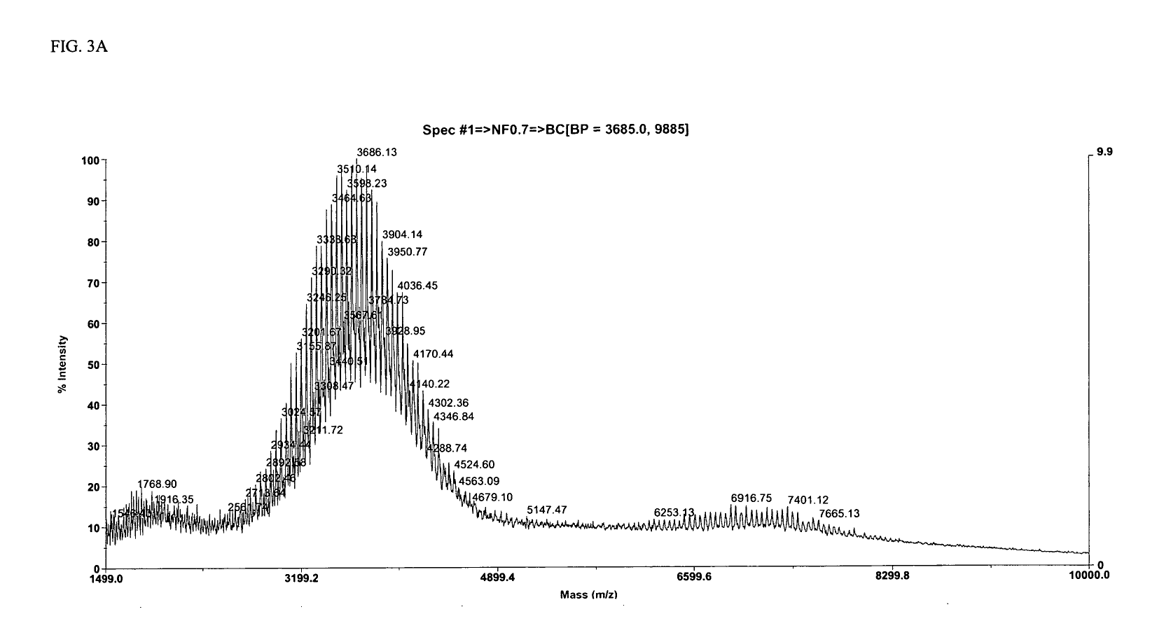

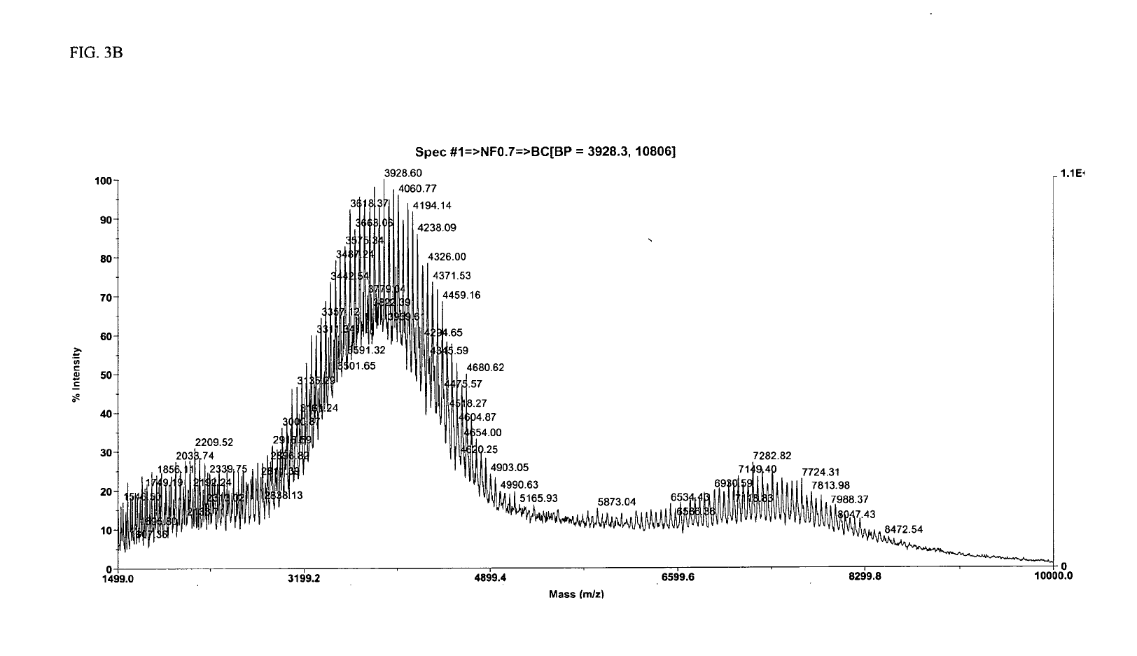

The structure of the intermediate conjugate 4 and the final conjugate 5 in FIG. 2A were confirmed by MALDI-MS using standard methods in the art, which showed agreement between the observed and calculated molecular weights. The results are presented in FIG. 3A for intermediate conjugate 4 and FIG. 3B for the final conjugate 5. Similar results were obtained for the intermediate conjugate 7 and the final conjugate 8 in FIG. 2B.

In the embodiments shown in FIGS. 2A and 2B, PEG serves as the solubilizing element. However, as discussed above other solubilizing elements may be used. Furthermore, while curcumin was used in this embodiment as the active ingredient, curcumin metabolites or curcumin analogues may be used as well.

FIG. 2C shows an alternate exemplary synthetic scheme for the preparation of one embodiment of a water soluble curcumin-based compound of the present disclosure. In this embodiment, a carbohydrate group serves as the solubilizing element. In this synthesis bis-(4-nitrophenyl carbonate) was reacted with curcumin (10) in the presence of diisopropylethylamine (DIEA) and tetrahydrofuran (THF) to form the intermediate conjugate 11 or 12. The intermediate conjugate was reacted with a carbohydrate, in this example D-2-deoxyglucosamine hydrochloride in the presence of DIEA and dimethylformamide (DMF) to form the final conjugate 13 or 14. In this embodiment, one molecule or more than one molecule of carbohydrate may be conjugated to curcumin.

The incorporation of the solubilizing element allows for improved water solubility of the water soluble curcumin-based compound. As a result of such increased water solubility, the active ingredient within the water soluble curcumin-based compound may be delivered more efficiently to a subject, resulting in a higher therapeutic index. Furthermore, the increased solubility will also lead to favorable formulation properties for the water soluble curcumin-based compounds. Such higher therapeutic index and favorable formulation properties may allow the use of such water soluble curcumin-based compounds in methods of treatment and/ore prevention that were not previously appreciated in the art.

### Pharmaceutical Compositions and Administration

The water soluble curcumin-based compound described in the present disclosure described above for use in the methods described herein may be administered alone or as a part of a pharmaceutical composition formulated by any method known in the art. Certain exemplary methods for preparing the pharmaceutical compositions are described herein and should not be considered as limiting examples. Furthermore, the water soluble curcumin-based compound or pharmaceutical compositions containing the water soluble curcumin-based compound may be administered to the subject as is known in the art and as determined by a healthcare provider. Certain modes of administration are provided herein and should not be considered as limiting examples. Furthermore, the water soluble curcumin-based compound or pharmaceutical compositions containing the water soluble curcumin-based compound may be administered with other agents in the methods described herein. Such other agents may be agents that increase the activity of the compounds disclosed, such as by limiting the degradation or inactivation of the compounds disclosed or increasing the absorption or activity of the compounds disclosed.

The pharmaceutical compositions containing the water soluble curcumin-based compound described can be used in the form of a medicinal preparation, for example, in aerosol, solid, semi-solid or liquid forms, which contains at least one water soluble curcumin-based compound disclosed as an active ingredient. In addition, the pharmaceutical compositions may be used in an admixture with an appropriate pharmaceutically acceptable carrier. Such pharmaceutically acceptable carriers include, but are not limited to, organic or inorganic carriers, excipients or diluents suitable for pharmaceutical applications. The active ingredient may be compounded, for example, with the usual non-toxic pharmaceutically acceptable carriers for tablets, pellets, capsules, inhalants, suppositories, solutions, emulsions, suspensions, aerosols and any other form suitable for use. Pharmaceutically acceptable carriers for use in pharmaceutical compositions are well known in the pharmaceutical field, and are described, for example, in Remington: The Science and Practice of Pharmacy Pharmaceutical Sciences, Lippincott Williams and Wilkins (A. R. Gennaro editor, 20^{th} edition). Such pharmaceutically acceptable carriers are nontoxic to the recipients at the dosages and concentrations employed and include, but are not limited to, water, talc, gum acacia, gelatin, magnesium trisilicate, keratin, colloidal silica, urea, buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, lactose, mannitol, glucose, mannose, dextrins, potato or corn starch or starch paste, chelating agents such as EDTA, sugar alcohols such as mannitol or sorbitol, counterions such as sodium and/or nonionic surfactants such as Tweens or Plurionics. In addition, the pharmaceutical compositions may comprise auxiliary agents, such as, but not limited to, taste-enhancing agents, stabilizing agents, thickening agents, coloring agents and perfumes.

Pharmaceutical compositions may be prepared for storage or administration by mixing a compound of the present disclosure having a desired degree of purity with physiologically acceptable carriers, stabilizers, auxiliary agents etc. as is known in the pharmaceutical field. Such pharmaceutical compositions may be provided in sustained release or timed release formulations.

The pharmaceutical compositions may be administered orally in solid dosage forms, such as capsules, tablets, and powders, or in liquid dosage forms, such as elixirs, syrups and suspensions. It can also be administered parenterally (such as by intramuscular or intravenous injection), in sterile liquid dosage forms. Furthermore, pharmaceutical compositions may be administered by transmucosal delivery via solid, liquid or aerosol forms of transdermally via a patch mechanism or ointment. Various types of transmucosal administration include respiratory tract mucosal administration, nasal mucosal administration, oral transmucosal (such as sublingual and buccal) administration and rectal transmucosal administration.

For preparing solid compositions such as, but not limited to, tablets or capsules, the pharmaceutical compositions may be mixed with an appropriate pharmaceutically acceptable carriers, such as conventional tableting ingredients (lactose, sucrose, mannitol, corn starch, potato starch, alginic acid, microcrystalline cellulose, acacia, gelatin, gums, colloidal silicon dioxide, croscarmellose sodium, talc, sorbitol, stearic acid magnesium stearate, calcium stearate, zinc stearate, stearic acid, dicalcium phosphate other excipients, colorants, diluents, buffering agents, disintegrating agents, moistening agents, preservatives, flavoring agents, and pharmacologically compatible carriers) and diluents (including, but not limited to, water, saline or buffering solutions) to form a substantially homogenous composition. The substantially homogenous composition means the components (a water soluble curcumin-based compound as described herein, a pharmaceutically acceptable carrier and auxiliary agents) are dispersed evenly throughout the composition so that the composition may be readily subdivided into equally effective unit dosage forms such as tablets, pills and capsules. The solid compositions described may be coated or otherwise compounded to provide a dosage form affording the advantage of prolonged action. For example, the tablet or pill can comprise an inner dosage an outer dosage component, the latter being in the form of an envelope over the former. The two components can be separated by an enteric layer which serves to resist disintegration in the stomach and permits the inner component to pass intact through the stomach or to be delayed in release. A variety of materials can be used for such enteric layers or coatings such materials including a number of polymeric acids and mixtures of polymeric acids with such materials as shellac, cetyl alcohol and cellulose acetate. The active compounds may also be formulated in rectal compositions such as suppositories or retention enemas, e.g., containing conventional suppository bases such as cocoa butter or other glycerides. The solid compositions may also comprise a capsule, such as hard- or soft-shelled gelatin type containing, for example, surfactants, lubricants, and inert fillers, such as lactose, sucrose, calcium phosphate, and corn starch.

For intranasal administration, intrapulmonary administration or administration by other modes of inhalation, the pharmaceutical compositions may be delivered in the form of a solution or suspension from a pump spray container or as an aerosol spray presentation from a pressurized container or nebulizer, with the use of a suitable propellant (e.g., dichlorodifluoromethane, trichlorofluoromethane, dichlorotetrafluoroethane, nitrogen, propane, carbon dioxide or other suitable gas) or as a dry powder. In the case of an aerosol or dry powder format, the amount (dose) of the compound delivered may be determined by providing a valve to deliver a metered amount.

Liquid forms may be administered orally, parenterally or via transmucosal administration. Suitable forms for liquid administration include aqueous solutions, suitably flavored syrups, aqueous or oil suspensions, and emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, sorbitol syrup, methylcellulose, polyvinylpyrrolidone or gelatin. Such liquid preparations may be prepared by conventional means with pharmaceutically acceptable additives such as suspending agents; emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners. Liquid formulations may include diluents, such as water and alcohols, for example, ethanol, benzyl alcohol, glycerin, and the polyethylene alcohols, either with or without the addition of a pharmaceutically acceptable surfactant, suspending agent, or emulsifying agent. For buccal or sublingual administration, the composition may take the form of tablets or lozenges formulated in conventional manners. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

The compounds disclosed (whether alone or in pharmaceutical compositions) may be formulated for parenteral administration. Parenteral administration includes, but is not limited to, intravenous administration, subcutaneous administration, intramuscular administration, intradermal administration, intrathecal administration, intraarticular administration, intracardiac administration, retrobulbar administration and administration via implants, such as sustained release implants. The requirements for effective pharmaceutically acceptable carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4th ed., 622-630 (1986).

The pharmaceutical compositions may be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The pharmaceutical compositions are administered in therapeutically effective amount. The therapeutically effective amount will, of course, vary depending upon known factors, such as the pharmacodynamic characteristics of the particular compound and its mode and route of administration; the age, health and weight of the subject; the severity and stage of the disease state or condition; the kind of concurrent treatment; the frequency of treatment; and the effect desired. The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or diseases, in particular chronic conditions or diseases, may require prolonged treatment involving multiple administrations.

### EXAMPLES

### Example 1

### Rate of Drug Release of Water Soluble Curcumin-Based Compounds

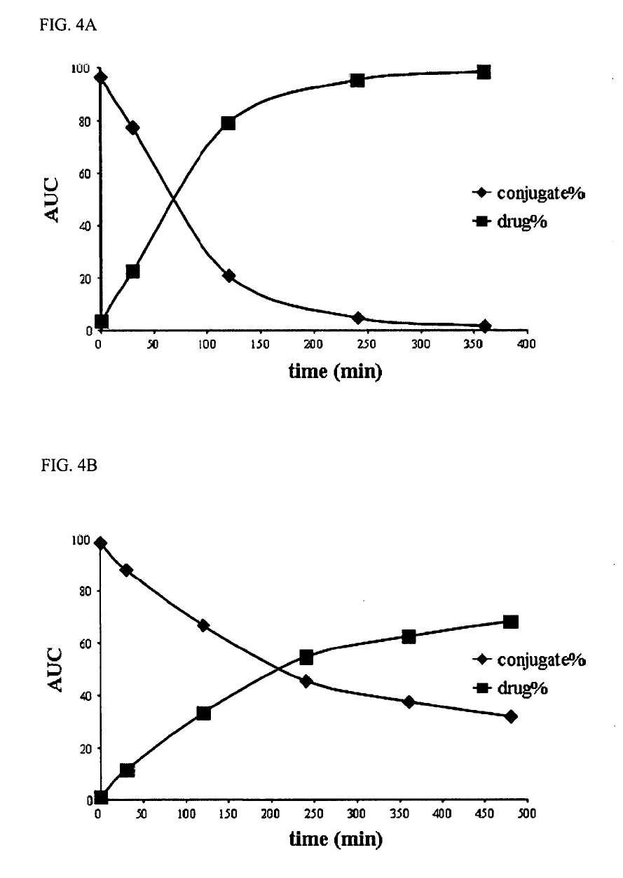

To investigate the stability of the drug-polymer bond and to determine the rate of drug release from the curcumin-PEG conjugate, conjugate solutions in phosphate-buffered saline (PBS, pH 7.4) were incubated at 37° C. Disappearance of the conjugate and formation of free curcumin were monitored by reverse phase (RP)-HPLC at 280 nm. The curcumin signal was identified by reference to an authentic curcumin sample. As shown in Table 1, the high and low molecular weight curcumin-PEG conjugates released the curcumin moiety at different rates. The high molecular weight conjugate (see FIG. 2A, compound 5) had a half-life of about 60 minutes and the low molecular weight conjugate (see FIG. 2B, compound 8) had a half-life of about 200 minutes. The conjugate decomposition patterns for compounds 5 and 8 are shown in FIG. 4A (compound 5) and FIG. 4B (compound 8). No precipitation of the water soluble curcumin-PEG conjugates was observed.

TABLE 1
Half-life (T_{1/2}) of curcumin release from water
soluble curcumin-based compounds^{a}

| Conjugate | Half-life (T_{1/2}) in min^{a} |
|---|---|
| Compound 5 | 60 |
| Compound 8 | 200 |

^{a}in PBS, pH 7.4 at 37° C.

### Example 2

### Effect of Water Soluble Curcumin-Based Compounds on the Growth of Human Prostate Cancer Cells

The growth inhibitory effects of conjugates 5 and 8 were examined through a series of cytoxcicity assays against a variety of cancer cell types of human origin. The cell types tested were PC-3 (prostate), LS-174T (colon), MIA PaCa-2 (pancreatic) and BxPC-3 (pancreatic). Experimental details are provided in the methods section.

In these experiments, curcumin (unmodified) was used as a reference compound. Curcumin and the water soluble curcumin-based compounds (conjugates 5 and 8) were dissolved in DMSO and water, respectively, prior to delivery; control cells received equal amounts of DMSO and culture medium. The cells were incubated in 24-well plates, and in separate groups, with DMSO alone (control), curcumin at concentrations of 5-20 μM, and water soluble curcumin-based compounds (conjugates 5 and 8) at concentrations of 5-20 μM, for 24 hours, at which time the cell culture media containing the compounds were removed by washing and aspiration and replaced with compound free cell culture media. The surviving cell populations were counted on the fourth day after treatment (96 hour post-treatment). The viable cells were then counted and the numbers of treated cells were normalized against the untreated controls (taken as 100%). The concentrations of curcumin are the actual curcumin concentrations in the conjugate, not the concentration of the conjugate itself. For example, the curcumin content of the water-soluble curcumin-based compound conjugate 5 is only 9.4%.

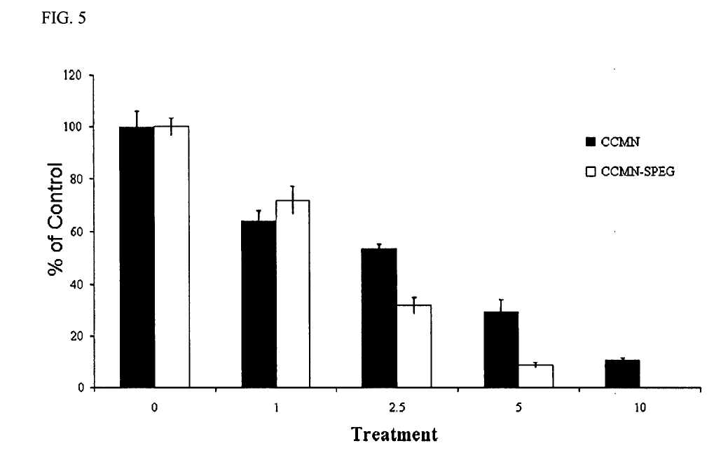

The results of these assays are shown in Table 2 below for conjugates 5 and 8. As can be seen, conjugates 5 and 8 exhibited equal or superior cytotoxic effects as compared to free curcumin against the cell lines tested. FIG. 5. shows a representative experiment illustrating the cytotoxic effects of conjugate 8 on BxPC-3 cells (pancreatic cancer). Again as illustrated in Table 2, conjugate 8 showed equal or superior cytotoxic effects as compared to free curcumin against the BxPC-3 cells.

TABLE 2
Cytotoxicity^{a} of curcumin and water-solubl curcumin-based
compounds against human carcinoma cell lines

| Treatment | PC-3 | LS-174T | MIA PaCa-2 | Bx-PC3 |
|---|---|---|---|---|
| Curcumin | 12.0 ± 1.35 | 6.5 ± 1.4 | 9.0 ± 2.5 | 2.0 ± 0.3 |
| Conjugate 5 | 5.0 ± 0.1 | ND^{b} | ND^{b} | ND^{b} |
| Conjugate 8 | 5.6 ± 0.2 | 4.0 ± 0.7 | 2.6 ± 0 | 2.1 ± 0.3 |

^{a}shown as IC50 (μM) ± standard error of the mean;
^{b}not determined

### Example 3

### Internalization of Water Soluble Curcumin-Based Compounds

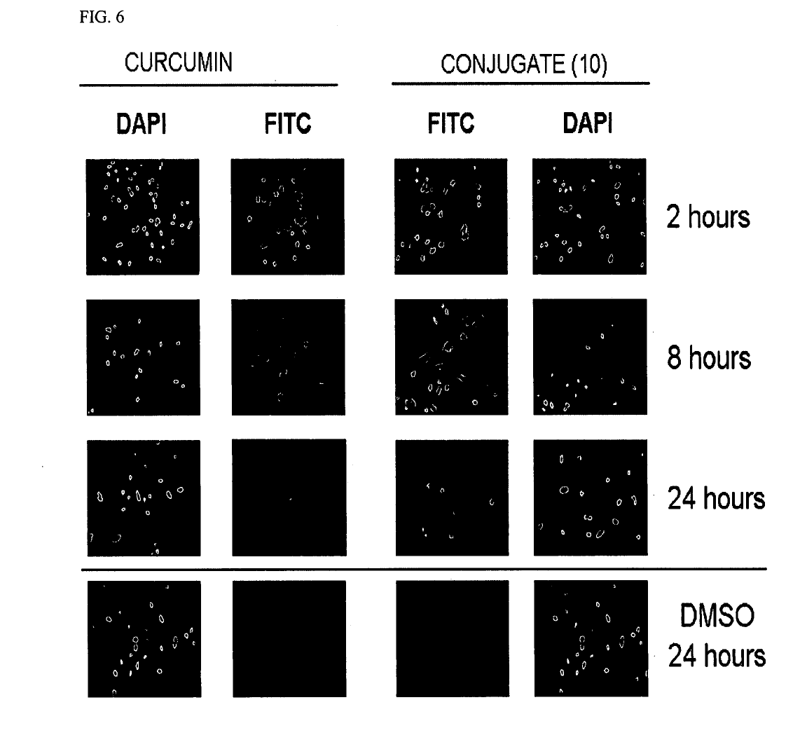

As demonstrated in Example 2 above, the water soluble curcumin-based compounds showed greater cytotoxicity than unmodified curcumin. The improvement in the conjugates' cytotoxicity is postulated to be due to their water solubility and cell internalization ability. Complete solubilization provides the cells to be treated with a longer “effective exposure time (EET)” to the curcumin contained in the water soluble curcumin-based compounds of the present disclosure. In contrast, the cells treated with unconjugated curcumin experienced a short EET due to a premature precipitation of curcumin. Therefore, the water solubility of the compounds of the present disclosure provides beneficial effects. Furthermore, the water soluble curcumin-based compounds of the present disclosure may also be internalized more efficiently than the unmodified curcumin. Such enhanced internalization also increases the activity of the water soluble curcumin-based compounds of the present disclosure. A facilitated internalization would be favorable to the cytotoxicity of curcumin as one of the mechanisms of action of this drug is inhibition of the nuclear factor KB. The internalization of the water soluble curcumin-based compounds and unmodified curcumin were examined using fluorescent microscopy in PC-3 cells. In this experiment, PC-3 cells were incubated with either DMSO, unmodified curcumin, or conjugate 8 in four-chamber microscope slides for a period of 2, 8 and 24 hours. At these time points, the PC-3 cells were fixed and viewed by fluorescent microscopy (FIG. 6). At all time points, the fluorescent emission intensities of the conjugate-treated cells were higher than those of cells treated with unmodified curcumin. In particular, at the 24 h time point, the curcumin emission was the same as that of the background, while that of the conjugate was visibly higher, indicating the presence of conjugate 8 in the nuclei. This experiment demonstrates that not only could both curcumin and the water soluble curcumin-based analogues undergo nuclear internalization, but the water soluble curcumin-based analogues had a prolonged internalization time, possibly due to a resistance to cellular efflux.

### Methods

### General

Reversed-phase (RP) HPLC was performed with a Beckman System Gold instrument operated by Beckman 32 Karat Version 5.0 software (Beckman Coulter, Fullerton, Calif.). Column: 4.6×250 mm, analytical C18 RP (GraceVydac, Hesperia, Calif.) column. Elution solvents: 0.1% TFA/water (solvent A) and a 10% to 90% gradient of 0.1:60:40, TFA:CH3CN:H2O, v/v, (solvent B). A solvent B gradient of 10%-90% was used in each run and within 20 min.

Methyl amino-PEG carboxylate was obtained from Nektar (Huntsville, Ala.). Curcumin and methoxy amino-PEG were purchased from Sigma-Aldrich-Fluka (Milwaukee, Wis.). All 1D proton NMR spectra were recorded on a Bruker Avance500 (500 MHz) spectrometer at 20° C. with 15 seconds recycle delay. An exponential window function with a line-broadening of 0.2 Hz was used on the time-domain data prior to Fourier transform. All the data collection and processing were done with Bruker XWINNMR 3.2 software. NMR analysis was not performed for the large conjugates due to difficulties in the complete removal of the water and solvent contaminations. They were, however, reliably identified by MALDI MS. Purities were tested with analytical RP-HPLC.

MALDI MS was performed in positive mode on a Voyager Elite mass spectrometer with delayed extraction technology (PerSeptive Biosystems, Framingham, Mass.). Sinapinic acid was used as matrix, and samples were prepared in a 50:50 (v/v) mixture of 0.1% TFA/acetonitrile. A 1-pmol/L solution of bovine serum albumin was added as internal standard.

Photo-absorption experiments were carried out in a Beckman model DU 640B spectrophotometer (Beckman Coulter, Fullerton, Calif.).

### Synthesis

Methyl N-(4-nitrophenyloxy carbonyl)amino-PEG³⁵⁰⁰-carboxylate, 4. Methyl amino-PEG carboxylate (2, Scheme 1, MW ˜3500) (104 mg, 0.03 mmol) in 10 mL of dry THF was added within 40 min to a solution of bis-(4-nitrophenyl)carbonate (3, 37.2 mg, 122.4 mmol) and DIEA (13.3 μL, 0.076 mmol) in 2 mL of dry THF. The mixture was stirred at room temperature (RT) under an argon atmosphere for 15 h. Additional portions of 3 (10.5 mg, 0.035 mmol) and DIEA (21 μL, 0.12 mmol) were added and stirring was continued for another 2.5 h.

The solvent was distilled in vacuum and the crude mixture was purified in a 2.5×25 Cm silica gel column, using 0%-20% methanol (MTL) gradient in chloroform (CHL) containing 0.2% HOAc to afford 81 mg (78%) of the pure product 4 as a highly viscous oil. Calculated MW: 3665; MALDI MS: 3686; RP-HPLC t_{R}: 21.4 min.

CCMNPEG³⁵⁰⁰-CO₂CH₃, 5. Compound 4 (75 mg, 0.022 mmol) was dissolved in 4 mL of dry DMF containing 17.4 μL (0.1 mmol) of DIEA. A solution of CCMN (36.8 mg, 0.1 mmol) in 2 mL of the same solvent was added and the mixture was stirred under argon and at RT for 3 days. The solvent was distilled in vacuum and the residue was redissolved in ethyl acetate (ETA) and was loaded into a 2.5×8.5 Cm silica gel column. The column was eluted with 20% hexanes in ETA, ETA, and then 5%-10% MTL/CHL containing 1% HOAc. Distillation of solvents afforded a solid. This was redissolved in 2 mL of distilled water and the slight quantity of fine particles was separated by filtration through a syringe-tip, 0.2 μm cellulose membrane. The clear solution was lyophilized to afford a bright yellow, water-soluble solid product in 67 mg (82%) yield. Calculated MW: 3894; MALDI MS: 3928; RP-HPLC t_{R}: 24.1 min.
Methoxy N-(4-nitrophenyloxy carbonyl)amino-PEG⁷⁵⁰, 7. The same reaction as for the preparation of 4, above, was employed using the methoxy amino-PEG⁷⁵⁰ (6, 980 mg, 1.3 mmol) as the starting compound. The crude product mixture was purified in a 2.5×10 Cm silica gel column, eluted with 0%-0.5% MTL/CHL to afford 1 g (83%) of a light yellow oil. Calculated MW: 916; MALDI MS: 906; RP-HPLC t_{R}: 19.5 min.
CCMNPEG⁷⁵⁰-OCH₃, 8. Compound 7 (500 mg, 0.55 mmol) was reacted with CCMN according to the procedure for the preparation of 5, above. Column chromatography on silica gel afforded 252 mg (40%) of the pure product as a deep red and highly viscous oil. Calculated MW: 1145; MALDI MS: 1145; RP-HPLC t_{R}: 24.25 min.

### Drug Release Kinetics

An authentic sample of curcumin was screened by RP-HPLC under the analytical conditions described in the General section, above, and showed a t_{R} of 25.7 min. A fresh sample of the conjugate was also chromatographed to serve as the t₀ reference profile. Conjugates were dissolved in 1× Dulbecco's phosphate-buffered saline and were incubated at 37° C. Aliquots were withdrawn at certain time intervals and were analyzed by HPLC, and the peaks of the forming curcumin and decomposing conjugate were integrated. At completion of the decomposition (no more change in the HPLC pattern), the % AUC for each compound was plotted against time on the same axes. The crossing point of the two curves determined the decomposition t_{1/2}. Averages of two runs were used to construct each plot.

### Water Solubility Test

Ten mg of curcumin was mixed with 1.0 mL of 18.2 MΩ water and the mixture was vortexed for 5 min, sonicated for 1 min, and centrifuged at 14,000 rpm for 5 min. The water layer was separated and scanned in a spectrophotometer at a wavelength range of 200-800 nm.

### Cytotoxicity Assays

Tumor cells (as indicated) were maintained as monolayers in 75-cm² tissue culture flasks using their respective cell culture medium containing 10% fetal bovine serum and 2 mM L-glutamine. Incubation was at 37° C. under a humidified 5% CO₂: air atmosphere (standard conditions) for five days. The cells were harvested when in mid-log growth and their concentration was determined using a particle counter (Beckman Coulter, Inc, Fullerton, Calif.). An aliquot of the cell suspension was diluted in culture medium for delivery to a 24-well tissue culture plate at a range of 10,000 to 30,000 per 1 mL per well. After 24 h, quadruplicate wells were inoculated with either vehicle (untreated controls), or test compound at various concentrations. After 24 h of incubation, the wells were aspirated, washed once with 1 ml PBS, then refilled with 1 ml treatment-free medium. Following a 96-h incubation under standard conditions from the initial treatment, the viable cells were counted and the numbers were normalized to the percent of untreated controls. The extent of cytotoxicity in treated wells as compared to the controls, and the dose that inhibits 50% cell proliferation (IC₅₀) was calculated using Microsoft Excel software program (Microsoft Corp., Redmond, Wash.).

### Fluorescent Microscopy

PC-3 cells were seeded into 4-chamber microscope slides. Treatments were added at a concentration of 40 μM in culture medium when the cells were approximately 70%-80% confluent. Curcumin and conjugate 10 were dissolved in DMSO and at equal volume. Two hours post-treatment (PT), the culture medium was removed, the cells were washed 4× with PBS, and fresh culture medium was added to the wells. At 2 h, 8 h, and 24 h PT, the cells were fixed in 3.7% aqueous formaldehyde for 15 min. The cells were washed 3× in PBS, then the nuclear DNA was stained with 4,6-diamidino-2-phenylindole (DAPI) for 5 min. The slides were preserved with Fluoromount G and coverslip. All images were taken with a Zeiss Axioplan Fluorescent Microscope using FITC filter for curcumin and conjugate 10, and DAPI filter for DAPI.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference. The appended claim is attached solely for the purposes of foreign priority, if required.

One skilled in the art will appreciate readily that the present disclosure is well adapted to carry out the objects and obtain the ends and advantages mentioned, as well as those objects, ends and advantages inherent herein. The present examples, along with the methods, procedures, treatments, molecules, and specific compounds described herein are representative of certain embodiments, are exemplary, and are not intended as limitations on the scope of the disclosure.

What is claimed:

1. (canceled)

2. A curcumin-based compound linked to at least one solubilizing agent, wherein the solubilizing element is selected from the group consisting of: poly(ethylene glycol) (PEG), derivatives of PEG, poly(substituted-2-oxazoline) (POZ), derivatives of POZ, an amino acid, a polypeptide and an antibody.

3. The compound of claim 2 where in the curcumin-based compound is selected from the group consisting of: Ar-tumerone, methylcurcumin, demethoxy curcumin, bisdemethoxycurcumin, sodium curcuminate, dibenzoylmethane, acetylcurcumin, feruloyl methanecurcumin, hexahydrocurcumin, tetrahydrocurcumin, 1,7-bis(4-hydroxy-3-methoxyphenyl)-1,6-heptadiene-3,5-dione (curcuminl), 1,7-bis(piperonyl)-1,6-heptadiene-3,5-dione(piperonyl curcumin)1,7-bis(2-hydroxy naphthyl)-1,6-heptadiene-2,5-dione(2-hydroxyl naphthyl curcumin) and 1,1-bis(phenyl)-1,3,8,10-undecatetraene-5,7-dione (cinnamyl curcumin).

4. The compound of claim 2 wherein the curcumin-based compound has the following structural formula 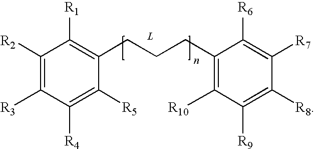
Wherein,
R₁ to R₁₀ are each independently selected from the group consisting of: hydrogen, alkyl, alkoxyl, acyl, hydroxyl, amino, alkylamino, dialkylamino, carboxyl, carbamoyl, thioacyl, sulfonyl, alkoxycarbonyl. alkyl amino carbonyl, dialkylaminocarbonyl, mercapto, alkylthio and salts and esters thereof;
L is an alkyl linking group and n=3-20.

5. The compound of claim 4 wherein R₁ to R₁₀ are each independently selected from the group consisting of: H, OH, NO₂, and OCH₃ and L has the following structural formula:

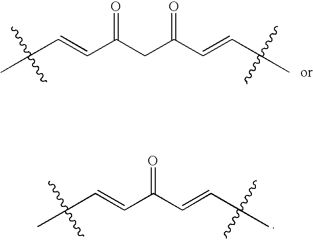

6. (canceled)

7. The compound of claim 2 where in the curcumin-based compound is selected from the group consisting of: dihydroferulic acid, ferulic acid and glucoronides of tetrahydrocurcumin and hexahydrocurcumin.

8. The compound of claim 2 wherein the solubilizing element is selected from the group consisting of: poly(ethylene glycol) (PEG), derivatives of PEG, poly(substituted-2-oxazoline) (POZ), and derivatives of POZ.

9. The compound of claim 2 wherein the solubilizing element is a PEG.

10. The compound of claim 2 where the curcumin-based compound is linked to the solubilizing agent by an amide, amine, ester, ether, thioether, sulfide, disulfide, hemiacetal, acetal, ketal, hydrazide, urethane or hydrazone linkage.

11. The compound of claim 2 where the curcumin-based compound is linked to the solubilizing agent by a urethane linkage.

12. The compound of claim 2 where the curcumin-based compound contains 2 or more solubilizing agents.

13. The compound of claim 2 where the curcumin-based compound contains 1 solubilizing agent.

14. The method of claim 9 where the PEG molecule has a molecular weight of 100 to 5000 Da.

15. (canceled)

16. (canceled)

17. (canceled)

18. A method of treating or preventing a disease state in a subject, said method comprising the step of administering to said subject a curcumin-based compound linked to at least one solubilizing agent, wherein the solubilizing element is selected from the group consisting of: poly(ethylene glycol) (PEG), derivatives of PEG., poly(substituted-2-oxazoline) (POZ), derivatives of POZ, an amino acid, a polypeptide and an antibody.

19. The method of claims 18 where the disease state is cancer or a chronic inflammatory condition.

20. (canceled)

21. (canceled)

22. (canceled)

23. The method of claim 18 wherein the solubilizing element is selected from the group consisting of: poly(ethylene glycol) (PEG), derivatives of PEG, poly(substituted-2-oxazoline) (POZ), and derivatives of POZ.

24. The method of claim 18 wherein the solubilizing element is a PEG.

25. The method of claim 18 where the curcumin-based compound is linked to the solubilizing agent by an amide, amine, ester, ether, thioether, sulfide, disulfide, hemiacetal, acetal, ketal, hydrazide, urethane or hydrazone linkage.

26. The method of claim 18 where the curcumin-based compound is linked to the solubilizing agent by a urethane linkage.

27. The method of claim 18 where the curcumin-based compound contains 2 or more solubilizing agents.

28. The method of claim 18 where the curcumin-based compound contains 1 solubilizing agent.

29. The method of claim 24 where the PEG molecule has a molecular weight of 100 to 5000 Da.

30. (canceled)

31. The method of claim 18 where in the curcumin-based compound is selected from the group consisting of Ar-tumerone, methylcurcumin, demethoxy curcumin, bisdemethoxycurcumin, sodium curcuminate, dibenzoylmethane, acetylcurcumin, feruloyl methanecurcumin, hexahydrocurcumin, tetrahydrocurcumin, 1,7-bis(4-hydroxy-3-methoxyphenyl)-1,6-heptadiene-3,5-dione (curcumin1), 1,7-bis(piperonyl)-1,6-heptadiene-3,5-dione(piperonyl curcumin)1,7-bis(2-hydroxy naphthyl)-1,6-heptadiene-2,5-dione(2-hydroxyl naphthyl curcumin) and 1,1-bis(phenyl)-1,3,8,10-undecatetraene-5,7-dione (cinnamyl curcumin).

32. The method of claim 18 wherein the curcumin-based compound has the following structural formula 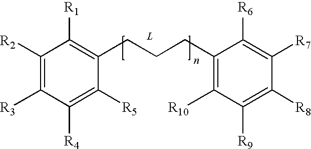
Wherein,
R₁ to R₁₀ are each independently selected from the group consisting of: hydrogen, alkyl, alkoxyl, acyl, hydroxyl, amino, alkylamino, dialkylamino, carboxyl, carbamoyl, thioacyl, sulfonyl, alkoxycarbonyl. alkyl amino carbonyl, dialkylaminocarbonyl, mercapto, alkylthio and salts and esters thereof;
L is an alkyl linking group and n=3-20.

33. The method of claim 32 wherein R₁ to R₁₀ are each independently selected from the group consisting of: H, OH, NO₂, and OCH₃ and L has the following structural formula:

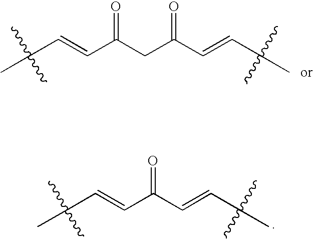

34. (canceled)

35. (canceled)

36. (canceled)

37. (canceled)

38. (canceled)

39. (canceled)

40. (canceled)

41. (canceled)

42. The method of claim 18 where the curcumin-based compound is selected from the group consisting of: dihydroferulic acid, ferulic acid and glucoronides of tetrahydrocurcumin and hexahydrocurcumin.

43. (canceled)

44. (canceled)

45. (canceled)

46. (canceled)

47. (canceled)

48. (canceled)

49. (canceled)

50. The compound of claim 2 where the curcumin-based compound has the following structural formula:

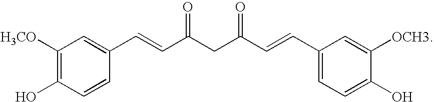

51. The method of claim 18 where the curcumin-based compound has the following structural formula:

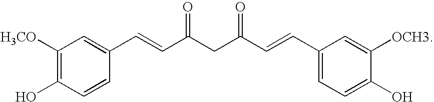

52. A pharmaceutical composition comprising a water soluble curcumin-based compound linked to at least one solubilizing agent, wherein the solubilizing element is selected from the group consisting of: poly(ethylene glycol) (PEG), derivatives of PEG, poly(substituted-2-oxazoline) (POZ), derivatives of POZ, an amino acid, a polypeptide and an antibody.
